(19) United States
(12) Patent Application Publication
(10) Pub. No.: US 2026/0118332 A1
(43) Pub. Date: Apr. 30, 2026

(54) VISIBLE HYDROGEN-CHROMIC SENSING MATERIAL, AND PREPARATION METHOD AND USE THEREOF

(71) Applicants: Nanjing University, Nanjing City (CN); Zhejiang Guwei Technology Co., Ltd., Hangzhou City (CN)

(72) Inventors: Peng Mao, Nanjing City (CN); Yutong Wu, Nanjing City (CN); Yuyuan Qin, Nanjing City (CN); Min Han, Nanjing City (CN); Fang Han, Nanjing City (CN)

(21) Appl. No.: 19/022,252

(22) Filed: Jan. 15, 2025

(30) Foreign Application Priority Data
Oct. 29, 2024 (CN) 202411517785.X

(51) Int. Cl. G01N 33/00; B01J 35/45; G01N 27/12; G01N 31/22

(52) U.S. Cl. CPC G01N 33/005; B01J 35/45; G01N 27/127; G01N 31/223

(57) ABSTRACT

Provided are a visible hydrogen-chromic sensing material, and a preparation method and use thereof. The visible hydrogen-chromic sensing material includes a substrate, a metal film layer, a semiconductor gas-sensitive film layer, and a nanocatalyst layer that are stacked in sequence.

### CROSS REFERENCE TO RELATED APPLICATION

This patent application claims the benefit and priority of Chinese Patent Application No. 202411517785X filed with the China National Intellectual Property Administration on Oct. 29, 2024, the disclosure of which is incorporated by reference herein in its entirety as part of the present application.

### TECHNICAL FIELD

The present disclosure relates to the technical field of gas sensing, and in particular to a visible hydrogen-chromic sensing material, and a preparation method and use thereof.

### BACKGROUND

Hydrogen, a key gas widely used in industry, energy and chemistry, is considered one of the ideal future energy sources because of its non toxicity, cleanliness, and renewability. However, hydrogen has a wide explosive limit in air (4 vol % to 74 vol %) and a low ignition temperature, which makes it prone to leakage and explosion accidents. Therefore, the detection and monitoring of hydrogen are of great significance. Existing hydrogen sensing technologies include electrochemical sensors, metal oxide semiconductor sensors, optical sensors, and the like. Hydrogen sensors based on metal oxide semiconductors (such as WO₃) have been widely studied and used due to their high sensitivity and selectivity. However, conventional metal oxide semiconductor sensors typically need to operate at a high temperature, which increases power consumption and use complexity of the sensors, thus limiting their application range.

In recent years, hydrogen-chromic materials have attracted widespread attention due to their ability to change color in the presence of hydrogen. This material not only enables the detection of hydrogen, but also allows visible detection through color changes, making it easy to operate and achieve rapid on-site detection. However, existing hydrogen-chromic materials are typically based on the intrinsic color of semiconductor gas-sensitive films, with very limited visibility.

### SUMMARY

In view of this, an object of the present disclosure is to provide a visible hydrogen-chromic sensing material, and a preparation method and the use thereof. The visible hydrogen-chromic sensing material exhibits a high degree of visibility.

In order to achieve the object described above, the present disclosure provides the following technical solutions.

The present disclosure provides a visible hydrogen-chromic sensing material, including a substrate, a metal film layer, a semiconductor gas-sensitive film layer, and a nanocatalyst layer that are stacked in sequence.

In some embodiments, the metal film layer has a thickness of 5 nm to 2,000 nm.

In some embodiments, an element in the metal film layer includes at least one selected from the group consisting of Cr, Mo, Ti, W, Au, Ag, Pt, Pd, Cu, and Al.

In some embodiments, the semiconductor gas-sensitive film layer has a thickness of 1 nm to 1,000 nm.

In some embodiments, the semiconductor gas-sensitive film layer includes at least one selected from the group consisting of a WO₃ film, a MoO₃ film, and a VO₂ film.

In some embodiments, the WO₃ film is doped with at least one element selected from the group consisting of Ti, Mo, and V, the MoO₃ film is doped with at least one element selected from the group consisting of Ti, W, and V, and the VO₂ film is doped with at least one element selected from the group consisting of Ti, W, and Mo.

In some embodiments, the nanocatalyst layer is a nanoparticle layer or a nanofilm layer, wherein nanoparticles in the nanoparticle layer have an average particle size of 1 nm to 100 nm, and the nanofilm has a thickness of 0.1 nm to 50 nm.

In some embodiments, an element in the nanocatalyst layer includes at least one selected from the group consisting of Pt, Pd, Au, Ag, Rh, Ru, Cu, Ti, and Ni.

The present disclosure further provides a method for preparing the visible hydrogen-chromic sensing material described in the technical solutions above, including:
depositing and forming the metal film layer, the semiconductor gas-sensitive film layer, and the nanocatalyst layer on a surface of the substrate in sequence to obtain the visible hydrogen-chromic sensing material.

The present disclosure further provides use of the visible hydrogen-chromic sensing material described in the technical solutions above in a field of gas sensors.

The present disclosure provides a visible hydrogen-chromic sensing material, including a substrate, a metal film layer, a semiconductor gas-sensitive film layer, and a nanocatalyst layer that are stacked in sequence.

Compared with the prior art, some embodiments of the present disclosure have the following beneficial effects:

### (1) Visible Detection

The visible hydrogen-chromic sensing material inherently possesses structural color, which originates from the coupling of plasmon modes of nanoparticles in the nanocatalyst layer and cavity modes in the semiconductor-metal film in the material. This strong coupling results in the material having selective light absorption and showing a specific color macroscopically. Moreover, the coupling modes in the material are highly sensitive to changes in the dielectric constant of the semiconductor gas-sensitive film layer, and hydrogen absorption can cause changes in the dielectric constant of the semiconductor gas-sensitive film layer. Even slight changes in the dielectric constant can lead to significant changes in the structural color of the material, achieving a very significant macroscopic visibility of the color change before and after hydrogen absorption, which facilitates directly visible detection. In addition, the visible hydrogen-chromic sensing material has rich and colorful structural colors. Unlike existing semiconductor hydrogen-chromic films, the visible hydrogen-chromic sensing material itself has a high degree of visibility of the structural color, and the color change thereof before and after hydrogen absorption varies significantly, resulting in a high degree of visibility. Furthermore, the structural color can be adjusted by adjusting the material parameters, such as the thickness or dielectric constant of the semiconductor gas-sensitive film layer.

### (2) Short Response Time

In conventional hydrogen-chromic films, the color-change mechanism primarily comes from hydrogen atoms diffusing into the semiconductor lattice, where semiconductor oxides grabs electrons of hydrogen atoms such that metal atoms in the semiconductor transition back and forth in different valences, resulting in light absorption. To achieve considerable light absorption and then lead to color change, it is necessary to allow sufficient hydrogen atoms to enter the lattice. The color-change mechanism of the visible hydrogen-chromic sensing material is the variation in the dielectric constant after hydrogen absorption of the semiconductor gas-sensitive film layer. A small number of hydrogen atoms entering the semiconductor gas-sensitive film layer can cause significant changes in the dielectric constant, and these changes occur rapidly, enabling the material to respond quickly to changes in hydrogen concentration. The hydrogen absorption and release time is short (within 10 s), allowing for real-time monitoring and observation.

### (3) High Sensitivity

Unlike the color-change mechanism of conventional hydrogen-chromic materials, the plasmon mode-cavity mode strong coupling supported in the hydrogen-chromic material is very sensitive to changes in the dielectric constant, which is the essence of the high sensitivity of the material, enabling the detection of hydrogen at concentrations as low as 0.001%.

### (4) Easy to Prepare on a Large Scale

The hydrogen-chromic sensing material is designed as a stacking structure and can be prepared by using standard thin-film deposition and spin-coating processes, making it suitable for large-scale production.

### (5) Convenient to use and Easy to Operate

The material has a simple structure and is easy to operate without complex equipment during detection.

The present disclosure further provides a method for preparing the visible hydrogen-chromic sensing material described in the above technical solutions. The method is simple and easy to realize industrial application.

In summary, the visible hydrogen-chromic sensing material of the present disclosure exhibits high-efficiency hydrogen-chromic performance and a high degree of visibility, can achieve a rapid and sensitive hydrogen detection. It is also easy to prepare on a large scale and to operate.

### BRIEF DESCRIPTION OF THE DRAWINGS

FIG. 1 shows a schematic structural diagram of the visible hydrogen-chromic sensing material of an embodiment of the present disclosure, where 1 refers to a substrate, 2 refers to a metal film layer, 3 refers to a semiconductor gas-sensitive film layer, and 4 refers to a nanocatalyst layer;

FIG. 2 shows a cross-sectional scanning electron microscope (SEM) morphology image of the visible hydrogen-chromic sensing material obtained in Example 1;

FIG. 3 shows a low-magnification scanning transmission electron microscopy (STEM) morphology image of Pt nanoparticles on the surface of the visible hydrogen-chromic sensing material obtained in Example 1;

FIG. 4 shows a high-magnification STEM morphology image of Pt nanoparticles on the surface of the visible hydrogen-chromic sensing material obtained in Example 1;

FIG. 5 shows reflectance spectra of the visible hydrogen-chromic sensing material obtained in Example 3 before and after introducing hydrogen;

FIG. 6 shows a response curve of the reflectivity at 700 nm of the visible hydrogen-chromic sensing material obtained in Example 1 as a function of the concentration of introduced hydrogen;

FIG. 7 shows a response curve of the reflectivity at 700 nm of the visible hydrogen-chromic sensing material obtained in Example 2 as a function of the concentration of introduced hydrogen;

FIG. 8 shows a response curve of the reflectivity at 700 nm of the visible hydrogen-chromic sensing material obtained in Example 3 as a function of the concentration of introduced hydrogen; and

FIG. 9A shows transmittance spectra of the visible hydrogen-chromic sensing material obtained in Comparative example 1 before and after introducing hydrogen, and FIG. 9B shows a response curve of the light absorptivity at 700 nm of the visible hydrogen-chromic sensing material obtained in Comparative example 1 as a function of the concentration of introduced hydrogen.

### DETAILED DESCRIPTION OF THE EMBODIMENTS

The present disclosure provides a visible hydrogen-chromic sensing material, including a substrate, a metal film layer, a semiconductor gas-sensitive film layer, and a nanocatalyst layer that are stacked in sequence.

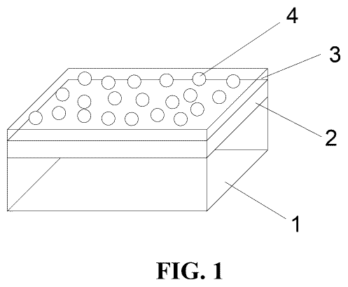

FIG. 1 shows a schematic structural diagram of the visible hydrogen-chromic sensing material of an embodiment of the present disclosure, where 1 refers to a substrate, 2 refers to a metal film layer, 3 refers to a semiconductor gas-sensitive film layer, and 4 refers to a nanocatalyst layer. The visible hydrogen-chromic sensing material will be described below with reference to FIG. 1.

The visible hydrogen-chromic sensing material of the present disclosure includes a substrate 1.

The thickness of the substrate 1 is not particularly limited in the present disclosure, and the substrate may have any thickness that is well known to those skilled in the art.

In some embodiments of the present disclosure, a material for the substrate 1 is selected from the group consisting of an insulator, a semiconductor, a silicon-on-insulator (SOI) material, a composite material of SOI with silicon and metals, a metal, and a polymer, where the insulator is selected from the group consisting of glass, quartz (SiO₂), and a ceramic, where the ceramic is alumina; the semiconductor is selected from the group consisting of silicon, GaN, and GaAs; the metal is selected from the group consisting of aluminum, a stainless steel, and an aluminum alloy; and the polymer is selected from the group consisting of silicone, polyvinyl alcohol (PVA), polyethylene terephthalate (PET), polyimide (PI), polyethylene naphthalate (PEN), polydimethylsiloxane (PDMS), and polytetrafluoroethylene (PTFE).

The visible hydrogen-chromic sensing material of the present disclosure includes a metal film layer 2.

In some embodiments of the present disclosure, the metal film layer 2 has a thickness of 5 nm to 2,000 nm, particularly 100 nm, 200 nm, 300 nm, 500 nm, 1,000 nm, 1,500 nm, or 2,000 nm.

In some embodiments of the present disclosure, an element in the metal film layer 2 includes at least one selected from the group consisting of Cr, Mo, Ti, W, Au, Ag, Pt, Pd, Cu, and Al.

In some embodiments of the present disclosure, a material for the metal film layer 2 is selected from the group consisting of an alloy and a metal mixture, where the alloy includes an alloy formed by two or more elements selected from the group consisting of Cr, Mo, Ti, W, Au, Ag, Pt, Pd, Cu, and Al, and the metal mixture includes a mixture formed by two or more selected from the group consisting of Cr, Mo, Ti, W, Au, Ag, Pt, Pd, Cu, and Al.

In some embodiments of the present disclosure, the semiconductor gas-sensitive film layer 3 has a thickness of 1 nm to 1,000 nm, particularly 100 nm, 200 nm, 300 nm, 350 nm, 500 nm, or 1,000 nm.

In some embodiments of the present disclosure, the semiconductor gas-sensitive film layer 3 includes at least one selected from the group consisting of a WO₃ film, a MoO₃ film, and a VO₂ film. Under a condition of the semiconductor gas-sensitive film layer 3 includes two or more selected from the group consisting of the WO₃ film, the MoO₃ film, and the VO₂ film, the semiconductor gas-sensitive film layer 3 is a composite gas-sensitive film, and the composite gas-sensitive film is preferably MoO₃-WO₃.

In some embodiments of the present disclosure, the WO₃ film is doped with at least one element selected from the group consisting of Ti, Mo, and V, the MoO₃ film is doped with at least one element selected from the group consisting of Ti, W, and V, and the VO₂ film is doped with at least one element selected from the group consisting of Ti, W and Mo. In the present disclosure, the content of the doping element in the semiconductor gas-sensitive film layer 3 is not particularly limited, and a content well known to those skilled in the art may be used.

In some embodiments of the present disclosure, the nanocatalyst layer 4 is a nanoparticle layer or a nanofilm layer, where nanoparticles in the nanoparticle layer has an average particle size of 1 nm to 100 nm, specifically 1 nm, 6 nm, 10 nm, 12 nm, 50 nm, or 100 nm, and the nanofilm layer has a thickness of 0.1 nm to 50 nm.

In some embodiments of the present disclosure, an element in the nanocatalyst layer 4 includes at least one selected from the group consisting of Pt, Pd, Au, Ag, Rh, Ru, Cu, Ti, and Ni.

In some embodiments of the present disclosure, a material for the nanocatalyst layer 4 is selected from the group consisting of an alloy and a metal mixture, where the alloy includes an alloy formed by two or more elements selected from the group consisting of Pt, Pd, Au, Ag, Rh, Ru, Cu, Ti, and Ni, and preferably Pt-Pd alloy nanoparticles, and the metal mixture includes a mixture formed by two or more selected from the group consisting of Pt, Pd, Au, Ag, Rh, Ru, Cu, Ti, and Ni.

The present disclosure further provides a method for preparing the visible hydrogen-chromic sensing material described in the technical solutions above, the method including the following step:
depositing and forming the metal film layer, the semiconductor gas-sensitive film layer, and the nanocatalyst layer on a surface of the substrate in sequence to obtain the visible hydrogen-chromic sensing material.

In some embodiments of the present disclosure, a deposition method for forming the metal film layer 2 includes at least one selected from the group consisting of physical vapor deposition, chemical vapor deposition, atomic layer deposition, and electroplating.

In some embodiments of the present disclosure, a deposition method for forming the semiconductor gas-sensitive film layer 3 includes at least one selected from the group consisting of physical vapor deposition, chemical vapor deposition, atomic layer deposition, sol-gel method, and solvothermal reaction method.

In some embodiments of the present disclosure, a deposition method for forming the nanocatalyst layer 4 includes at least one selected from the group consisting of physical vapor deposition, chemical vapor deposition, atomic layer deposition, solvent reaction method, and gas aggregation method.

The specific parameters for the depositing are not particularly limited in the present disclosure, and a manner well known to those skilled in the art may be used.

The present disclosure further provides use of the visible hydrogen-chromic sensing material described in the technical solutions above in a field of gas sensors.

The specific mode of the use is not particularly limited in the present disclosure, and any mode that is well known to those skilled in the art can be used.

The technical solutions of the present disclosure will be described clearly and completely below in connection with examples of the present disclosure. Apparently, the described examples are just some examples of the present disclosure, not all of them. Based on the examples in the present disclosure, all the other examples that would have been obtained by those of ordinary skill in the art without any inventive effort shall fall within the scope of the present disclosure.

### Example 1

A method for preparing a visible hydrogen-chromic sensing material was performed as follows:

(1) Deposition of a metal film layer on a surface of a substrate: quartz (SiO₂) was selected as the substrate, and a Pt metal film layer was then deposited on the surface of the substrate by using electron beam evaporation, with a film thickness of 100 nm.

(2) A semiconductor gas-sensitive film layer was then deposited on a surface of the Pt metal film layer by using electron beam evaporation, where the semiconductor gas-sensitive film layer was WO₃, with a film thickness of 200 nm.

(3) A nanocatalyst layer was deposited on a surface of the semiconductor gas-sensitive film layer WO₃ by using gas cluster beam deposition, where the nanocatalyst layer was composed of Pt nanoparticles with an average size of 6 nm, that is the nanocatalyst layer having a thickness of 6 nm.

### Example 2

A method for preparing a visible hydrogen-chromic sensing material was performed as follows:

(1) Deposition of a metal film layeron a surface of a substrate: silicon was selected as the substrate, and a Ag metal film layer was then deposited on the surface of the substrate by using thermal evaporation, with a film thickness of 200 nm.

(2) A semiconductor gas-sensitive film layer was then deposited on a surface of the Ag metal film layer by using electron beam evaporation, where the semiconductor gas-sensitive film layer was TiₓWO₃, with a film thickness of 300 nm.

(3) A nanocatalyst layer was deposited on a surface of the semiconductor gas-sensitive film layer TiₓWO₃ by using gas cluster beam deposition, where the nanocatalyst layer was composed of Pd nanoparticles with an average size of 10 nm, that is the nanocatalyst layer having a thickness of 10 nm.

### Example 3

A method for preparing a visible hydrogen-chromic sensing material was performed as follows:

(1) Deposition of a metal film layer on a surface of a substrate: PET was selected as the substrate, and a Al metal film layer was then deposited on the surface of the substrate by using magnetron sputtering, with a film thickness of 300 nm.

(2) A semiconductor gas-sensitive film layer was then deposited on a surface of the Al metal film layer by using electron beam evaporation, where the semiconductor gas-sensitive film layer was a MoO₃-WO₃ composite film including a MoO₃ film having a thickness of 100 nm and a WO₃ film having a thickness of 250 nm, with the total thickness of the MoO₃-WO₃ composite film being 350 nm.

(3) A nanocatalyst layer was deposited on a surface of the semiconductor gas-sensitive film layer, i.e., the MoO₃-WO₃ composite film, by using gas cluster beam deposition, where the nanocatalyst layer was composed of Pt-Pd alloy nanoparticles with an average size of 12 nm, that is the nanocatalyst layer having a thickness of 12 nm.

### Comparative Example 1

Quartz was selected as a substrate, a WO₃ film was prepared on a surface of the substrate by using electron beam evaporation, and Pt nanoparticles were then deposited on a surface of the WO₃ film by using gas cluster beam deposition, where the WO₃ film had a thickness of 200 nm, and the Pt nanoparticles had an average size of 6 nm. The Comparative example 1 was the same as Example 1, except that no Pt metal film was prepared on the surface of the quartz substrate.

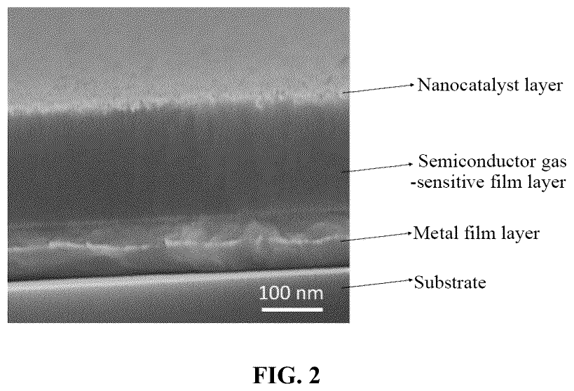

FIG. 2 shows an SEM cross-sectional morphology image of the visible hydrogen-chromic sensing material obtained in Example 1. As can be seen, a Pt metal film layer is provided on a surface of a SiO₂ substrate, a WO₃ semiconductor gas-sensitive film layer is provided on a surface of the Pt metal film layer, and a Pt nanocatalyst layer is provided on a surface of the WO₃ semiconductor gas-sensitive film layer.

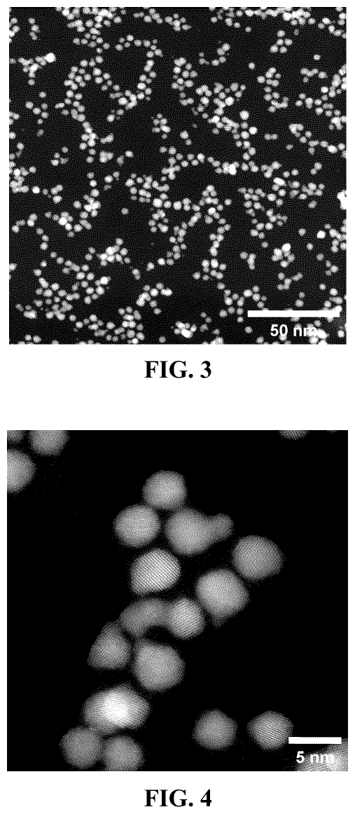

FIG. 3 shows a low-magnification STEM image of the Pt nanocatalyst layer in Example 1. As can be seen, the metal nanoparticle catalyst layer is composed of Pt nanoparticles having an average size of 6 nm.

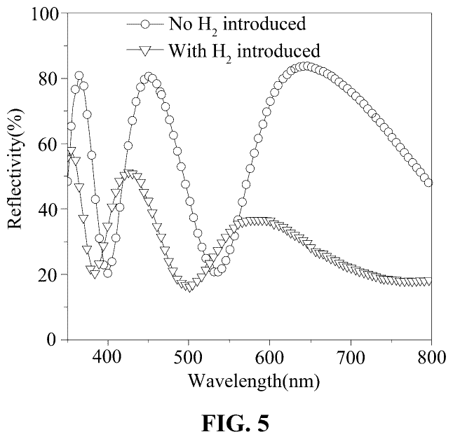

FIG. 4 shows a high-magnification STEM image of the Pt nanoparticle catalyst layer in Example 1. As can be seen, the Pt nanoparticle catalyst layer exhibits good monodispersity, and it can be clearly seen from the lattice fringes that Pt nanoparticles are in a crystalline state.

The visible hydrogen-chromic sensing materials obtained in Examples 1 to 3 and Comparative example 1 were placed in a test chamber, and an air-hydrogen mixed gas was introduced, with the gas pressure in the chamber being maintained at 1 atmosphere. The concentration of hydrogen was arbitrarily adjusted by controlling the flow rate of the gas flowmeter. The reflectance and transmission spectra and the hydrogen response curves at single wavelength were all measured by a grating spectrometer.

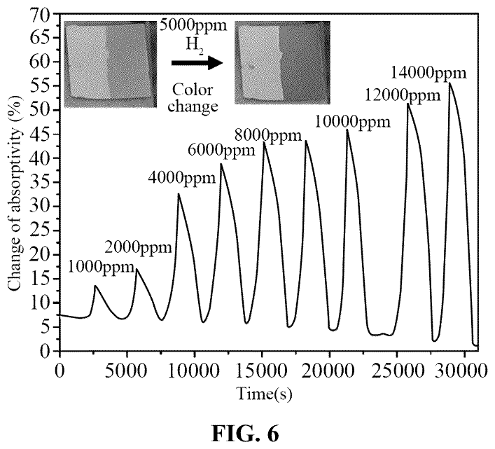

FIG. 5 shows the reflectance spectra of the visible hydrogen-chromic sensing material of Example 3 before and after introducing a hydrogen-air mixed gas with a hydrogen concentration of 2000 ppm. From the spectra, it can be seen that both the reflection peak and absorption peak of the material undergo a significant blue shift after the hydrogen-air mixed gas is introduced, and correspondingly, the absorption of the material is also increased significantly after the introduction of hydrogen.

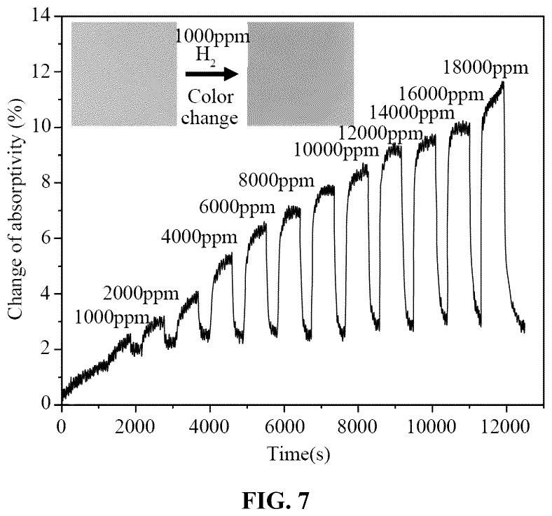

FIG. 6 shows a response curve of the single-wavelength light absorptivity at a wavelength of 700 nm of the visible hydrogen-chromic sensing material obtained in Example 1 as a function of the concentration of hydrogen, With the concentration of hydrogen varying in a range of 1,000 ppm to 14,000 ppm, the color of the material also correspondingly changes significantly from green to dark violet. The inset shows the actual hydrogen-chromic effect of the material when 5,000 ppm of hydrogen is introduced. It can be seen that the color-change effect of the hydrogen-chromic material is noticeable, with a high degree of visibility.

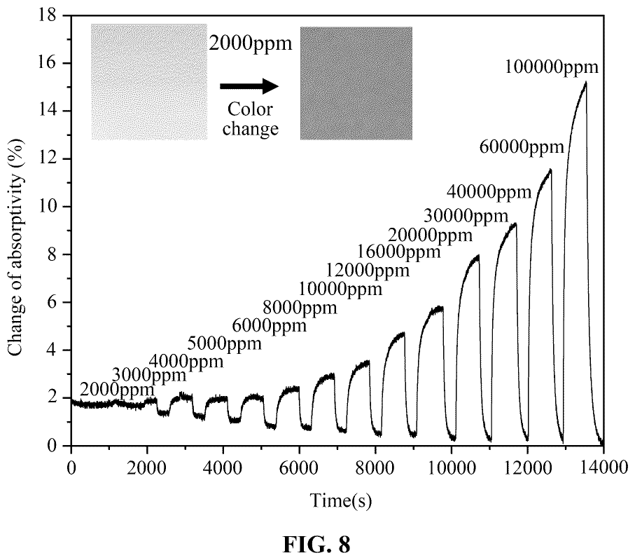

FIG. 7 shows a response curve of the single-wavelength light absorptivity at a wavelength of 700 nm of the visible hydrogen-chromic sensing material obtained in Example 2 as a function of the concentration of hydrogen. With the concentration of hydrogen varying in a range of 1,000 ppm to 18,000 ppm, the color of the material also correspondingly changes significantly from red to yellow. The inset shows the actual hydrogen-chromic effect of the material when 1,000 ppm of hydrogen is introduced. It can be seen that the color-change effect of the hydrogen-chromic material is noticeable, with a high degree of visibility.

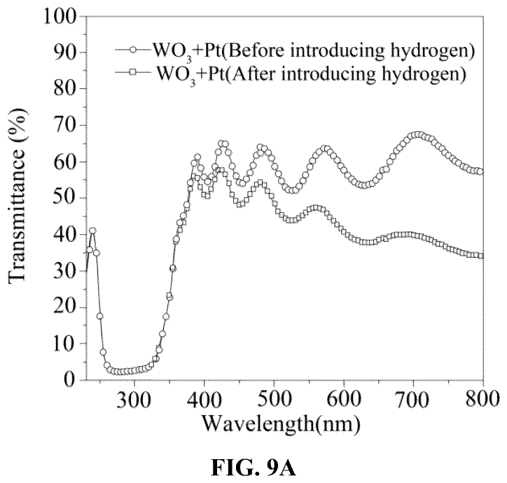

FIG. 8 shows a response curve of the single-wavelength light absorptivity at a wavelength of 700 nm of the visible hydrogen-chromic sensing material obtained in Example 3 as a function of the concentration of hydrogen. With the concentration of hydrogen varying in a range of 2,000 ppm to 10,000 ppm, the color of the material also correspondingly changes significantly from light violet to dark violet. The inset shows the actual hydrogen-chromic effect of the material when 2,000 ppm of hydrogen is introduced. It can be seen that the color-change effect of the hydrogen-chromic material is noticeable, with a high degree of visibility.

Comparing the visible hydrogen-chromic sensing materials obtained in Examples 1 to 3, it can be found that the preparation parameters of the materials are different, such that the resulting visible hydrogen-chromic sensing materials have different structural colors, and the difference in the structural colors results from different light absorptions of the materials. The light absorption of the visible hydrogen-chromic sensing material of the present disclosure is mainly adjusted and controlled by the thickness of the semiconductor gas-sensitive film layer. The thickness of the semiconductor gas-sensitive film layers is different, the light absorption bands of the materials are different, and correspondingly the color of the materials is also different. However, regardless of the preparation parameters, the materials prepared show significant hydrogen chromism after hydrogen being introduced, with a high degree of visibility. This also reflects from another aspect that the hydrogen-chromic material of the present disclosure is less dependent on the accuracy of the structural parameters thereof and thus is easy to prepare, because regardless of the parameters used to prepare materials, although the initial structural color is different, a hydrogen-chromic effect with high degree of visibility can be obtained. In addition, as can be seen from the response curve of the single-wavelength light absorption as a function of the concentration of hydrogen, the hydrogen-chromic materials of the present disclosure all exhibit a significant change in light absorption over a wide hydrogen concentration range (1,000 ppm to 100,000 ppm). This also means that the material is very suitable for preparing hydrogen sensors based on light absorption.

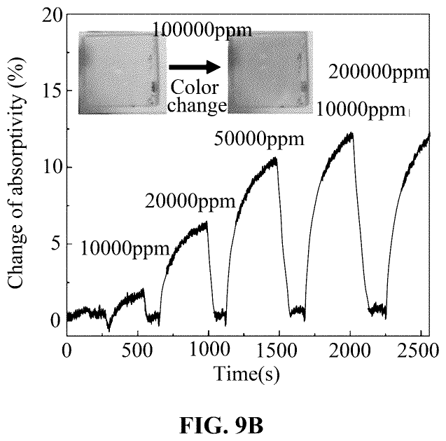

FIG. 9A shows transmittance spectra of a conventional material with good hydrogen-chromic performance prepared in Comparative example 1 before and after introducing hydrogen (the concentration of the introduced hydrogen is 100,000 ppm). It can be seen that the material itself is transparent and does not have the structural color as the hydrogen-chromic materials in Examples 1 to 3 of the present disclosure. The material obtained in Comparative example 1 shows a significant decrease in transmittance after hydrogen is introduced, and the decrease in the transmittance is more visible especially in the longer wavelength range of 600 nm to 800 nm. This indicates that the absorption of the material is increased after hydrogen is introduced, and the transparency of the material decreases, resulting in a certain degree of visibility. From the inset in FIG. 9B, it can be seen that the material does undergo hydrogen chromism, but the color change effect is poor with low degree of visibility. FIG. 9B shows the response curve of the single-wavelength light absorptivity at a wavelength of 700 nm of the hydrogen-chromic material obtained in Comparative example 1 as a function of the concentration of hydrogen, with the concentration of hydrogen varying in a range of 10,000 ppm to 200,000 ppm. It can be seen that when the light absorption changes significantly, the concentration of hydrogen is as high as 10,000 ppm, which is much different from the hydrogen-chromic material of the present disclosure.

The above descriptions are merely preferred embodiments of the present disclosure and are not intended to limit the present disclosure in any form. It should be noted that, for those of ordinary skills in the art, various modifications and improvements can be made without departing from the principle of the present disclosure, which should also be construed as falling within the scope of the present disclosure.

1. A visible hydrogen-chromic sensing material, comprising a substrate, a metal film layer, a semiconductor gas-sensitive film layer, and a nanocatalyst layer that are stacked in sequence.

2. The visible hydrogen-chromic sensing material of claim 1, wherein the metal film layer has a thickness of 5 nm to 2,000 nm.

3. The visible hydrogen-chromic sensing material of claim 1, wherein an element in the metal film layer comprises at least one selected from the group consisting of Cr, Mo, Ti, W, Au, Ag, Pt, Pd, Cu, and Al.

4. The visible hydrogen-chromic sensing material of claim 1, wherein the semiconductor gas-sensitive film layer has a thickness of 1 nm to 1,000 nm.

5. The visible hydrogen-chromic sensing material of claim 1, wherein the semiconductor gas-sensitive film layer comprises at least one selected from the group consisting of a WO₃ film, a MoO₃ film, and a VO₂ film.

6. The visible hydrogen-chromic sensing material of claim 5, wherein the WO₃ film is doped with at least one element selected from the group consisting of Ti, Mo, and V,
the MoO₃ film is doped with at least one element selected from the group consisting of Ti, W, and V, and
the VO₂ film is doped with at least one element selected from the group consisting of Ti, W, and Mo.

7. The visible hydrogen-chromic sensing material of claim 1, wherein the nanocatalyst layer is a nanoparticle layer or a nanofilm layer,
wherein nanoparticles in the nanoparticle layer have an average particle size of 1 nm to 100 nm, and
the nanofilm layer has a thickness of 0.1 nm to 50 nm.

8. The visible hydrogen-chromic sensing material of claim 1, wherein an element in the nanocatalyst layer comprises at least one selected from the group consisting of Pt, Pd, Au, Ag, Rh, Ru, Cu, Ti, and Ni.

9. A method for preparing the visible hydrogen-chromic sensing material of claim 1, comprising:
depositing and forming the metal film layer, the semiconductor gas-sensitive film layer, and the nanocatalyst layer on a surface of the substrate in sequence to obtain the visible hydrogen-chromic sensing material.

10. The visible hydrogen-chromic sensing material of claim 2, wherein an element in the metal film layer comprises at least one selected from the group consisting of Cr, Mo, Ti, W, Au, Ag, Pt, Pd, Cu, and Al.

11. The visible hydrogen-chromic sensing material of claim 4, wherein the semiconductor gas-sensitive film layer comprises at least one selected from the group consisting of a WO₃ film, a MoO₃ film, and a VO₂ film.

12. The visible hydrogen-chromic sensing material of claim 7, wherein an element in the nanocatalyst layer comprises at least one selected from the group consisting of Pt, Pd, Au, Ag, Rh, Ru, Cu, Ti, and Ni.

13. The method of claim 9, wherein the metal film layer has a thickness of 5 nm to 2,000 nm.

14. The method of claim 9, wherein an element in the metal film layer comprises at least one selected from the group consisting of Cr, Mo, Ti, W, Au, Ag, Pt, Pd, Cu, and Al.

15. The method of claim 9, wherein the semiconductor gas-sensitive film layer has a thickness of 1 nm to 1,000 nm.

16. The method of claim 9, wherein the semiconductor gas-sensitive film layer comprises at least one selected from the group consisting of a WO₃ film, a MoO₃ film, and a VO₂ film.

17. The method of claim 16, wherein the WO₃ film is doped with at least one element selected from the group consisting of Ti, Mo, and V,
the MoO₃ film is doped with at least one element selected from the group consisting of Ti, W, and V, and
the VO₂ film is doped with at least one element selected from the group consisting of Ti, W, and Mo.

18. The method of claim 9, wherein the nanocatalyst layer is a nanoparticle layer or a nanofilm layer,
wherein nanoparticles in the nanoparticle layer have a average particle size of 1 nm to 100 nm, and
the nanofilm layer has a thickness of 0.1 nm to 50 nm.

19. The method of claim 9, wherein an element in the nanocatalyst layer comprises at least one selected from the group consisting of Pt, Pd, Au, Ag, Rh, Ru, Cu, Ti, and Ni.

20. The method of claim 13, wherein an element in the metal film layer comprises at least one selected from the group consisting of Cr, Mo, Ti, W, Au, Ag, Pt, Pd, Cu, and Al.
